(19) United States
(12) Patent Application Publication
(10) Pub. No.: US 2026/0063413 A1
(43) Pub. Date: Mar. 5, 2026

(54) Swept source OCT system with multi spatial mode gain chip

(71) Applicant: KineoLabs, Inc., Billerica, MA (US)

(72) Inventors: J. Grant Houston, Lexington, MA (US); Walid A. Atia, Jamaica Plain, MA (US)

(21) Appl. No.: 19/306,565

(22) Filed: Aug. 21, 2025

Related U.S. Application Data
(60) Provisional application No. 63/687,513, filed on Aug. 27, 2024.

(51) Int. Cl. G01B 9/02091; A61B 3/10; G01B 9/02; G01B 9/02004; G01B 9/02056

(52) U.S. Cl. CPC G01B 9/02091; A61B 3/102; G01B 9/02004; G01B 9/02056; G01B 9/02083

(57) ABSTRACT

A full-field or line-field swept-source optical coherence tomography (OCT) system that uses a tilt-tuned cat's-eye laser whose semiconductor gain chip is dimensioned to lase in multiple spatial modes. The multimode output is preserved by free-space or multimode-fiber coupling from the laser to the interferometer, and is shaped by cylindrical line-forming optics to illuminate the sample with a long aspect-ratio line or across the field. The multimode operation produces a super-Gaussian, near flat-top intensity profile along the line or field and reduces spatial coherence, improving detector uniformity and lowering pixel cross-talk. Example implementations use a single-angled-facet gain chip with ridge width >3 μm and/or active-layer ridge height >2 μm, a thin-film interference filter tilt-scanned by a servoed galvanometer with encoder, and a line-scan camera to acquire parallel A-scans for B-scan formation. The approach maintains OCT advantages while relaxing single-mode constraints on the swept source and improving line-field and full-field image quality.

### RELATED APPLICATIONS

This application claims the benefit under 35 USC 119 (c) of U.S. Provisional Application No. 63/687,513, filed on Aug. 27, 2024, which is incorporated herein by reference in its entirety.

### BACKGROUND OF THE INVENTION

Optical coherence tomography (OCT) is a cross-sectional, non-invasive imaging modality that is used in many areas of medical imaging. For example, in ophthalmology, OCT has been widely used for imaging the retina, choroid and anterior segment. Functional imaging of the blood velocity and vessel microvasculature is also possible.

Fourier-domain OCT (FD-OCT) has recently attracted more attention because of its high sensitivity and imaging speed compared to time-domain OCT (TD-OCT), which uses an optical delay line for mechanical depth scanning with a relatively slow imaging speed. The spectral information discrimination in FD-OCT is accomplished either by using a dispersive spectrometer in the detection arm (spectral domain or SD-OCT) or rapidly scanning a swept laser source (swept-source OCT or SS-OCT).

Compared to spectrometer-based FD-OCT, swept-source OCT (SS-OCT) has several advantages, including its robustness to motion artifacts and fringe washout, lower sensitivity roll-off and higher detection efficiency.

Many different high-speed swept source architectures have been proposed for SS-OCT. One approach employs a semiconductor optical amplifier (SOA) based ring laser design (see for example Yun et al. “High-speed optical frequency-domain imaging” Opt. Express 11:2953 2003 and Huber et al. “Buffered Fourier domain mode locking: unidirectional swept laser sources for optical coherence tomography imaging at 370,000 lines/s,” Opt. Express 13, 3513 2005). Short cavity lasers (see for example Kuznetsov et al. “Compact Ultrafast Reflective Fabry-Perot Tunable Lasers For OCT Imaging Applications,” Proc. SPIE 7554: 75541F 2010) are another example. SOA based ring laser designs have been practically limited to positive wavelength sweeps (increasing wavelength) because of the significant power loss that occurs in negative tuning. This has been attributed to four-wave mixing (FWM) in SOAs causing a negative frequency shift in intracavity light as it propagates through the SOA (Bilenca et al. “Numerical study of wavelength-swept semiconductor ring lasers: the role of refractive-index nonlinearities in semiconductor optical amplifiers and implications for biomedical imaging applications,” Opt. Lett. 31:760-762 2006).

At the same time, other architectures exist for SS-OCT that reduce the performance requirements for the swept laser source. Fechtig et al. in an article entitled Line-Field parallel swept source MHz OCT for structural and functional retinal imaging, Biomedical Optics Express 716, vol. 6, no. 3, (2015) describes a system that achieves 1 MHz equivalent A-scan rates by combining a lower sweep rate laser with a linear sensor. Even earlier examples exist such as Line-Field Optical Coherence Tomography Using Frequency-Sweeping Source by Lee et al. in IEEE Journal of Selected Topics in Quantum Electronics, Vol. 14, No. 1, January 2008.

### SUMMARY OF THE INVENTION

The present invention concerns a tunable laser for a line-field swept source OCT system and the system itself that supports higher order spatial modes for better power distribution and lower spatial coherence over the extent of the line projected on the patient or other sample. The multimode output is preserved to the OCT interferometer and formed into a line illumination that exhibits a super-Gaussian, near flat-top intensity distribution and reduced spatial coherence across the line, thereby improving detector uniformity and reducing pixel cross-talk in line-field OCT.

In another aspect, the swept source that deliberately supports multiple spatial modes is employed in a full-field parallel OCT architecture that acquires an entire areal field on a two-dimensional (2-D) camera at each sampled wavenumber or frequency during the sweep. Preserving the multiple spatial modes from the source to the interferometer produces (i) a super-Gaussian-like, near flat-top areal irradiance distribution over the two dimensional field of view and (ii) reduced spatial coherence across both lateral axes, which suppresses pixel-to-pixel cross-talk, coherent fixed-pattern artifacts, and etalon fringes that are otherwise exacerbated by single-mode illumination in full-field OCT. The multimode swept source thus improves areal uniformity and image quality while retaining the robustness and depth-gating advantages of swept-source OCT.

In one aspect, an optical coherence tomography (OCT) system is provided that includes a swept optical source having a semiconductor gain chip configured during operation to lase in multiple spatial modes, including at least one higher-order mode beyond the fundamental, coupling optics that deliver the source output to an interferometer while preserving the multiple spatial modes, and a free-space interferometer with free-space reference and sample arms. The system illuminates a sample and recombines the sample and reference light for detection to obtain interferometric signals from which depth-resolved information is reconstructed.

The spatial mode-preserving coupling between the swept source and the interferometer can be realized by a free-space relay and/or by a multimode optical fiber chosen to maintain higher-order spatial content rather than filtering it to a single mode. In some examples, a multimode fiber of ≥1 m, ≥10 m, or ≥40 m in length is used to tailor lateral spatial coherence delivered to the interferometer. NA/aperture matching and core size selection are used so that the coupling does not spatially filter the higher-order content.

Preserving higher-order spatial modes from the source to the interferometer yields a less-peaked illumination profile at the sample (e.g., a super-Gaussian/near flat-top distribution) and reduces lateral spatial coherence across the illuminated field. These effects improve uniformity of signal-to-noise across the sensor and mitigate pixel cross-talk and coherent fixed-pattern artifacts compared with single-mode illumination.

The detector architecture is flexible: the interferometric output can be sensed by a linear pixel array for line-field parallel OCT or by a two-dimensional pixel array for full-field parallel OCT. In either case, the free-space interferometer provides the reference and sample paths, and mode-preserving coupling maintains the multimode field delivered to the interferometer.

The swept source is architecture-agnostic: the optical tuning mechanism can include an interference filter, grating, MEMS, or other tunable element. In some embodiments, a tilt-tuned thin-film interference filter is driven by a servo with encoder feedback along a prescribed tuning curve, but the benefits of multimode generation and mode-preserving delivery are independent of the specific tuner.

The semiconductor gain chip can be dimensioned or otherwise configured to support higher-order spatial modes, for example by selecting ridge geometries (e.g., ridge width W>3 μm and/or active-layer ridge height H>2 μm) that increase lateral and/or transverse mode order, although other structures may be used.

In representative implementations suited to ophthalmic imaging, the source may operate near 840 nm with a tuning range of 30-100 nm, enabling detection on silicon imagers; however, the approach applies across other wavelength bands and material systems. The free-space interferometer may incorporate the usual sample-arm and reference-arm optics (e.g., ocular compensation in the sample arm and path-length control in the reference arm) without departing from the present concepts.

The degree of lateral coherence delivered to the interferometer can be engineered by combinations of (i) gain-chip mode count (e.g., via ridge geometry), (ii) multimode fiber length/aperture, and (iii) free-space relay stops, enabling a trade-off between speckle statistics, artifact suppression, and fringe contrast while preserving the axial coherence needed for OCT ranging.

Another aspect provides a method of OCT that includes generating a swept beam with a multimode semiconductor source, delivering the beam to a free-space interferometer through mode-preserving coupling, dividing the beam into free-space reference and sample arms, interfering the beams at a detector, and reconstructing depth-resolved information. The method emphasizes preservation of higher-order spatial modes between the source and interferometer, which underlies the illumination uniformity and reduced cross-talk benefits described above.

The above and other features of the invention including various novel details of construction and combinations of parts, and other advantages, will now be more particularly described with reference to the accompanying drawings and pointed out in the claims. It will be understood that the particular method and device embodying the invention are shown by way of illustration and not as a limitation of the invention. The principles and features of this invention may be employed in various and numerous embodiments without departing from the scope of the invention.

### BRIEF DESCRIPTION OF THE DRAWINGS

In the accompanying drawings, reference characters refer to the same parts throughout the different views. The drawings are not necessarily to scale; emphasis has instead been placed upon illustrating the principles of the invention. Of the drawings:

FIG. 1 is a schematic view of a line-field OCT system including its swept source to which the invention is applicable;

FIGS. 2A, 2B, 2C, and 2D are schematic top views of gain chips for the swept laser source;

FIGS. 3A and 3B are schematic front elevational views of gain chips for the swept source illustrating different emission patterns associated with multimode operation;

FIG. 4 is a top view of the interferometer of the line-field OCT system according to one implementation; and

FIG. 5 is a plot of laser power along the major axis of the line or along both axes of a two dimensional field as rendered on the retina 204 of the patient's eye 202.

### DETAILED DESCRIPTION OF THE PREFERRED EMBODIMENTS

The invention now will be described more fully hereinafter with reference to the accompanying drawings, in which illustrative embodiments of the invention are shown. This invention may, however, be embodied in many different forms and should not be construed as limited to the embodiments set forth herein; rather, these embodiments are provided so that this disclosure will be thorough and complete, and will fully convey the scope of the invention to those skilled in the art.

As used herein, the term “and/or” includes any and all combinations of one or more of the associated listed items. Also, all conjunctions used are to be understood in the most inclusive sense possible. Thus, the word “or” should be understood as having the definition of a logical “or” rather than that of a logical “exclusive or” unless the context clearly necessitates otherwise. Further, the singular forms and the articles “a”, “an” and “the” are intended to include the plural forms as well, unless expressly stated otherwise. It will be further understood that the terms: includes, comprises, including and/or comprising, when used in this specification, specify the presence of stated features, integers, steps, operations, elements, and/or components, but do not preclude the presence or addition of one or more other features, integers, steps, operations, elements, components, and/or groups thereof. Further, it will be understood that when an element, including component or subsystem, is referred to and/or shown as being connected or coupled to another element, it can be directly connected or coupled to the other element or intervening elements may be present.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

As used herein, the parallel-detection OCT encompasses both line-field and full-field configurations. In line-field systems a one-dimensional line is illuminated and sensed in parallel by a linear array; in full-field systems a two-dimensional area is illuminated and sensed in parallel by a 2-D camera. Unless expressly stated otherwise, features described with respect to the line-field embodiment apply, mutatis mutandis, to the full-field embodiment, including the swept laser architecture, interferometer topology, and servo/encoder synchronization used to reference optical frequency during the sweep (see encoder 160, PID 164, and interferometer 205).

Full-field illumination refers to an areal pattern (e.g., rectangular or circular) whose aspect ratio is ≤10:1, and usually ≤4:1 and often ≤4:1 and whose linear dimensions are selected to cover the desired field of view on the sample (e.g., 1-12 mm for ophthalmic retina). “Reduced spatial coherence across the field” denotes a lateral coherence width at the sample plane that is comparable to, or smaller than, several camera pixels, which attenuates long-range interference across the area while preserving the axial coherence properties required for OCT.

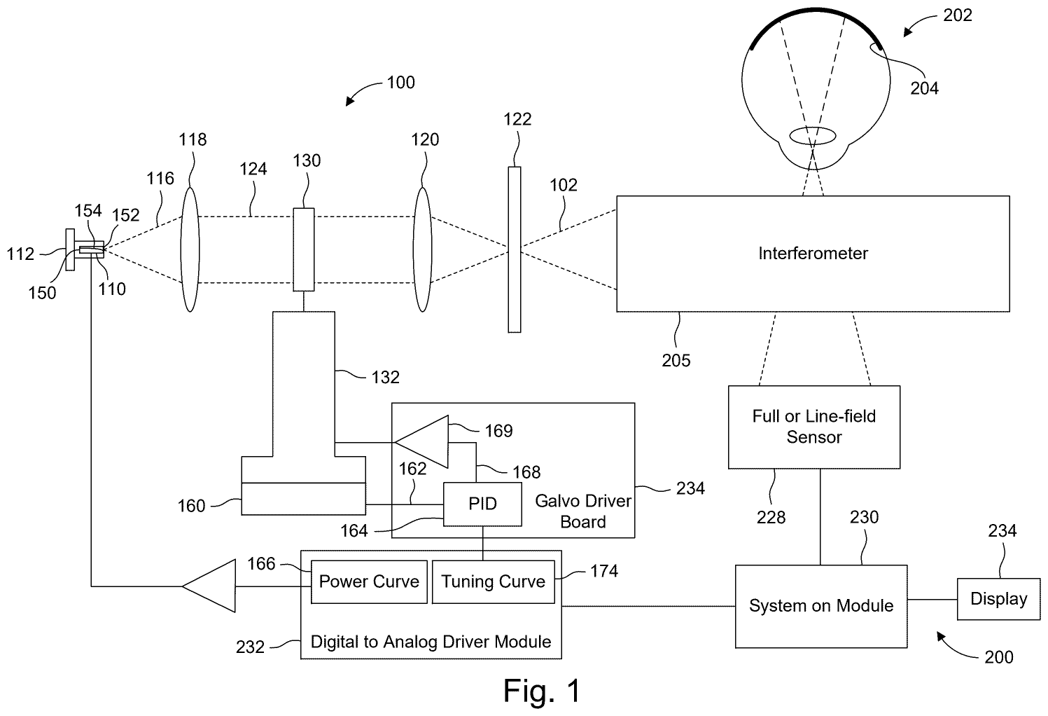

FIG. 1 shows a line-field parallel swept OCT system 200 with a cat's-eye tunable laser swept source 100 coupled to a line-scan or line-field sensor 228 via interferometer 205.

The laser's amplification is provided by a GaAlAs gain chip 110, in one example. This exemplary gain chip 110 amplifies light in the wavelength range of about 800 to 900 nanometers. Preferably its center wavelength is around 840 nanometers, which is useful for applications such as ophthalmic imaging and other diagnostic uses because of the water window (650 to 950 nm) at these wavelengths. Another advantage of this wavelength range is that it can be detected with standard cameras with silicon-based imager chips. Specifically, the output is detected with silicon, e.g., complementary metal-oxide-semiconductor (CMOS) or charge-coupled device (CCD), imagers.

Nevertheless, other material systems can be selected for the gain chip according to other examples. Common material systems are based on III-V semiconductor materials, including binary materials, such as GaN, GaAs, InP, GaSb, InAs, as well as ternary, quaternary, and pentenary alloys, such as InGaN, InAlGaN, InGaP, AlGaAs, InGaAs, GaInNAs, GaInNAsSb, AlInGaAs, InGaAsP, AlGaAsSb, AlGaInAsSb, AlAsSb, InGaSb, InAsSb, and InGaAsSb. Collectively, these material systems support operating wavelengths from about 400 nanometers (nm) to 2500 nm, including longer wavelength ranges extending into multiple micrometer wavelengths. Semiconductor quantum well, quantum cascade and quantum dot gain regions are typically used to obtain especially wide gain and spectral emission bandwidths, and support operation up to 250 μm in wavelength. Quantum well layers may be purposely strained or unstrained depending on the exact materials and the desired wavelength coverage.

In the preferred current embodiment, the gain chip 110 is mounted in a TO-can type hermetic package 112. This protects the chip 110 from dust and the ambient environment including moisture. In some examples, the TO-can package has an integrated or a separate thermoelectric cooler.

The chip 110 is preferably a single angled facet (SAF) edge-emitting chip. As such, it has a high reflectivity (HR) coated rear facet 150. It has an antireflective (AR) coated front facet 152. Its curved ridge waveguide 154 is perpendicular to the rear facet 150 but is angled at the interface with the front facet 152. This angling at the front facet along with the AR coating reduces reflections at the front facet reflectivity by up to 40 dB and significantly improves laser performance by reducing parasitic reflections that can otherwise lead to non-smooth tuning and mode-hopping.

The free space beam 116 from the package 112 is diverging in both axes (x, y). It is collimated by a collimating lens 118. The resulting collimated beam 124 is received by a cat's eye focusing lens 120, which focuses the light onto a cat's eye mirror/output coupler 122. This defines the other end of the laser cavity, extending between the mirror/output coupler 122 and the back/reflective facet of the gain chip 110.

The collimated light 124 between the collimating lens 118 and the cat's eye focusing lens 120 passes through a thin film interference bandpass filter 130. This provides a pass band of approximately 0.3 nanometers (nm) full width at half maximum (FWHM) for OCT applications. More generally, its pass band is between 0.2 nm and 0.5 nm FWHM, or more generally between 0.1 nm and 2 nm FWHM. Even more generally, it is between 0.05 nm to 5 nm FWHM.

The bandpass filter is held on an arm of an angle control actuator 132 that changes the angle of the bandpass filter 130 to the collimated light 124. Generally, the angle is modulated over a range of greater than 10 or 20 degrees, and typically greater than 30 degrees, and often up to about 35 degrees. Currently, the angle is changed between 110 degrees to about 130-140 degrees or more, measured between the plane of the filter 130 and the axis of the beam 124. In one example, the angle control actuator is a galvanometer. In other examples, the angle control actuator 132 is a servomotor or an electrical motor that continuously spins the bandpass filter 130 in the collimated beam 124. This allows for tilting of the bandpass filter 130 with respect to the collimated beam 124 to thereby tilt-tune the filter and thus change the passband to scan or sweep the wavelength of the swept laser 100.

Tuning speed specifications for a galvanometer generally range from 0.1 Hz to 50 kHz. For the higher speeds, a 25 kHz resonant galvanometer can be used with bi-directional tuning, but higher and lower speeds can be used. Wavelength tuning speed is usually given in nm/sec, so for a 100 Hz tuning speed ideal for retinal imaging applications where a line-speed camera at 100 kHz will give 1000 sampled bandwidth points and 70 nm tuning range, this would give 70 nm/10 msec=7000 nm/sec. In general, the tuning speed should be between 3,000 nm/sec and 11,000 nm/sec or higher.

For retinal or industrial imaging with low-cost CMOS or CCD cameras, 840 nm center wavelength is an ideal water window. The tuning range is usually minimally 30 nm of tuning range. Preferably, the tuning range is closer to 60 nm or 70 nm or more. This provides good resolution of <8 micrometers in air. In general, the tuning range should be between 30 nm and 100 nm.

The size of the collimated beam 124 in the laser's cavity is important for many applications. As a general rule, a smaller beam results in higher divergence resulting in a larger cone half angle (CHA). This reduces the minimum line width over angle for a tunable filter. In the current embodiment, the collimated beam is preferably not less than, i.e., greater than, 1 millimeter (mm) FWHM in diameter and is preferably greater than 2 mm FWHM for retinal OCT application. Its diameter can be smaller, however, for many spectroscopy applications in the infrared, visible or ultraviolet. In general, the CHA should be ≤0.04°×0.02°, and preferably ≤0.02°×0.01°.

The light from the gain chip is polarized. In the common architectures, the polarization is horizontal or parallel to the epitaxial layers of the edge-emitting gain chip 110. In the one configuration, the filter is oriented to receive the S polarization in order to maintain narrow line width of the filter as it is tilt tuned. On the other hand, the P polarization broadens somewhat at large tilt angles. S polarization has higher loss at larger tilt angles than P. So, the filter design needs to address these issues by providing a low enough loss across the tuning band for S, in the current embodiment. However, in some examples, the P polarization is used to provide higher power.

In general, the present cat's-eye configuration provides a number of advantages. It provides low loss, low tolerance, repeatable stable operation since it provides for a lower angle wavelength change over grating-based lasers.

The mirror/output coupler 122 will typically reflect about 80% of the light back into the laser's cavity and transmit about 20% of light. More generally, the mirror/output coupler can reflect from 10% to 99% of light (transmitting 90% to 1%, respectively), depending on the output power and laser cavity loss desired. Higher reflectivity results in lower loss cavities and thus wider laser tuning range where gain exceeds loss, but results in lower output power. In typical operation, the mirror/output coupler 122 reflects less than 90%.

Here, the diverging beam 102 from the mirror output coupler 122 is sent to the interferometer 205.

There are other ways of extracting the light from the laser cavity. A beam splitter can also be located in the cavity on either side of the interference filter 130. Light can also be extracted from the back/reflective facet of the gain chip 110 by using a chip coating with a lower reflectivity.

One characteristic of the beam 102 from the laser 100 is that it exhibits higher order spatial modes. Typically, such modes, even if present in the laser's cavity, are stripped out by intervening single mode fiber. However, in the current embodiment, these higher order spatial modes in the beam are preserved by the free space coupling between the laser 100 and the interferometer 205. However, in other embodiments, the multimode laser is coupled to the interferometer 205 via multimode optical fiber. In this way, the typically Gaussian power roll off associated with single spatial mode beams is avoided or at least partially mitigated. Instead, the present system provides a more top hat beam profile or a more consistent power profile across the extent of the beam 102 and most importantly along the line that will be projected on the patient's retina.

Nevertheless, in some examples, multimode fiber is used between the laser and the interferometer. In one embodiment, a long length of multimode fiber is used to couple the laser 100 to the interferometer. This multimode fiber is preferably over 1 meter, and preferably over 10 meters in length and possibly as long as 40 meters in length or more. This multimode fiber functions to reduce the spatial coherence across the beam to thereby lower cross talk between pixels of the line field or scan sensor 228.

The angle control actuator 132 is operated as a servomechanism. In the illustrated embodiment, the angle control actuator 132 is a servo controlled galvanometer with an encoder 160. The encoder 160 produces an angle signal 162 indicating the angle of the galvanometer and thus the filter 130 to the collimated beam 124. Preferably, the encoder is an optical encoder and is often analog.

The galvanometer 132 is operated by a galvo driver board 234 that receives the angle signal 162. A PID (proportional-integral-derivative) controller 164 implemented on the galvo driver board 234 compares the instantaneous angle signal 162 to a desired angle dictated by a tuning curve 174. The PID controller 164 produces the control function 168 that is used to drive the windings of the galvanometer 132 via an amplifier 169.

In the illustrated example, the OCT system 200 is employed for ophthalmic analysis of a human eye 202 and specifically the retina 204. That said, the system can also be used for analysis of other parts of the body such as the anterior chamber of the eye and/or other samples, both living and non-living, including industrial uses.

Light in the form of free space beam 102 from the laser 100 passes to interferometer 205 that couples light between line-scan sensor 228 and the sample 202 such as a patient's eye.

The line field sensor typically has a linear array of at least 512 pixels, and often has at least 1024 or 2048 pixels to detect interference signals for a line. In a current example, the linear array is a few pixels wide such as between 2 and 10 pixels wide. Often the pixels can be binned in this lateral axis for higher sensitivity.

In the current implementation, the OCT system 200 is controlled by a single board computer 230. Specifically, it is System on Module (SOM) that includes a graphic processing unit (GPU), central processing unit (CPU), memory, power management, high-speed interfaces. Currently a Jetson Orin series module is used from NVIDIA Corporation.

The SOM 230 controls a digital to analog driver module 232 which principally controls the drive to the chip 110 and the angle control actuator/galvanometer 132. In more detail, the digital to analog driver module 232 includes a tuning curve module 174 that stores a specified tuning function for the angle of the filter 130. This is supplied to the PID controller 164, which tries to minimize the error between the angle signal 162 and the tuning curve across the wavelength sweep of the laser 100. Often, the desired tuning curve is stored in a look up table or is generated algorithmically. Often this is an approximately sawtooth or triangular waveform.

In some embodiments, a power-vs-angle feed-forward profile stored in a power curve module controls the injection current to the chip 110 to be synchronized to the measured encoder angle so that chip current is modulated as a function of instantaneous filter tilt. The feed-forward reduces sweep-to-sweep power variation and maintains substantially constant output power or desired power shape across the tuning range.

The output from the line field sensor 228 is readout by the SOM 230. The results can be stored in the SOM 230 and/or displayed on display 234. The Fourier transform of the interference light is performed by the GPU within the SOM 230 at the different wavelengths or frequencies of the swept laser 100 reveals the profile of scattering intensities at different path lengths, and therefore scattering as a function of depth (z-direction or axial direction) in the sample (see for example Leitgeb et al., “Ultrahigh resolution Fourier domain optical coherence tomography,” Optics Express 12(10): 2156 2004). The profile of scattering as a function of depth for a point is called an axial scan (A-scan). The combination of the projected line and line-scan sensor 228 produces a cross-sectional image (tomogram or B-scan) of the sample.

While the previous description has focused in a line-field system, the principles can be extended to a full field system. In a full-field parallel swept-source OCT system, the tunable cat's-eye laser 100 and servo-encoded tuning (160, 164) are as shown in FIG. 1, but the line-forming optics are replaced by field-forming optics that expand and homogenize the beam to uniformly illuminate a 2-D area on the sample. The interferometer 205 combines a field-expanded reference beam and a field-expanded sample beam so that the entire area interferes on a 2-D camera (replacing the line-field sensor 228. The system-on-module (SOM) 230 uses the encoder-derived wavelength angle to register each camera frame to its instantaneous wavenumber position during the sweep.

The multimode output of laser 100 is preserved to the interferometer via free space or multimode fiber so that the areal illumination exhibits a near flat-top spatial profile and lower lateral coherence. This flattening and coherence reduction mitigate bright-spotting, vignetting-induced SNR falloff, and coherent fixed-pattern artifacts on the 2-D camera.

The SOM 230 synchronizes the camera exposure start/stop to the encoder 160 such that a burst of frames is captured over each sweep (or over both directions for bidirectional tuning). These frames sample the sweep in k-space and are Fourier transformed along the wavenumber dimension to reconstruct depth for each camera pixel, yielding an areal en face depth map or a volume when multiple areas are tiled.

In full-field implementations, the same multimode-preserving coupling described above is employed—e.g., a multimode fiber of ≥1 m, ≥10 m, or >40 m length, or a free-space relay—so that spatial coherence across the 2-D field is reduced. This lowers coherent cross-talk between non-adjacent camera pixels and suppresses parasitic interference from weak stray reflections within the field optics.

In some examples, a weak angular or diffusive homogenizer (e.g., a low-scatter ground-glass plate or engineered diffuser operated at low diffusion angles) is added in a collimated section of the path to spatially mix the multimode field while maintaining the instantaneous spectral linewidth required for axial OCT ranging. The homogenizer is preferably used in combination with the multimode source so that power throughput remains high and illumination remains temporally coherent for OCT sweeping.

The degree of lateral coherence at the sample plane can be adjusted by the multimode fiber length, mode count of the chip (ridge width/height), and the aperture stops of the field optics, enabling a design trade-off between speckle statistics, artifact suppression, and fringe contrast on the camera.

In the full-field embodiment, the detector or sensor 228 is a two-dimensional camera (global-shutter CMOS preferred) with ≥512×512 active pixels, often ≥1024×1024, 10-12-bit depth, and burst-mode readout synchronized to the sweep. Smaller regions of interest may be used at higher frame rates to increase the number of k-samples per sweep.

The 2-D camera acquires N frames over a sweep (e.g., 256-2048 frames per sweep), each tagged by the encoder angle (or derived k-index). After dark/flat corrections, the SOM 230 performs per-pixel resampling to linear k, followed by a 1-D Fourier transform along the frame axis to reconstruct A-lines per pixel, forming an en face depth map or 3-D volume.

In some embodiments, the reference beam is given a small off-axis tilt (e.g., ≤5° relative to the sample beam at the camera) so that the interferometric cross-term is shifted in spatial frequency on the sensor, enabling numerical separation of DC/auto-terms prior to k-space processing.

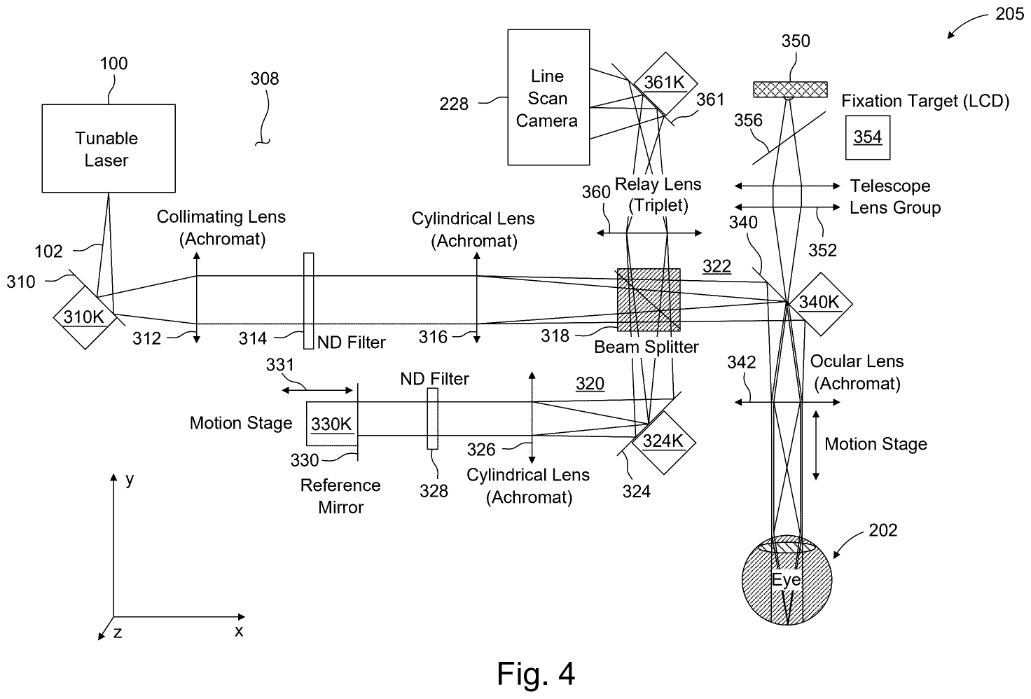

Alternatively, a phase-stepping approach is used in which the reference mirror 330 (mounted on linear motion rail 331 and driven by actuator 331A in FIG. 4) is dithered by sub-wavelength steps (e.g., π/2 increments) across successive frames at a fixed wavelength, permitting complex-field retrieval that can improve full-field reconstruction.

The SOM 230 (e.g., Jetson Orin) executes the resampling, Fourier transforms, phase retrieval (if used), and visualization, streaming en face planes at selected depths or rendering volumes in real time on display 234.

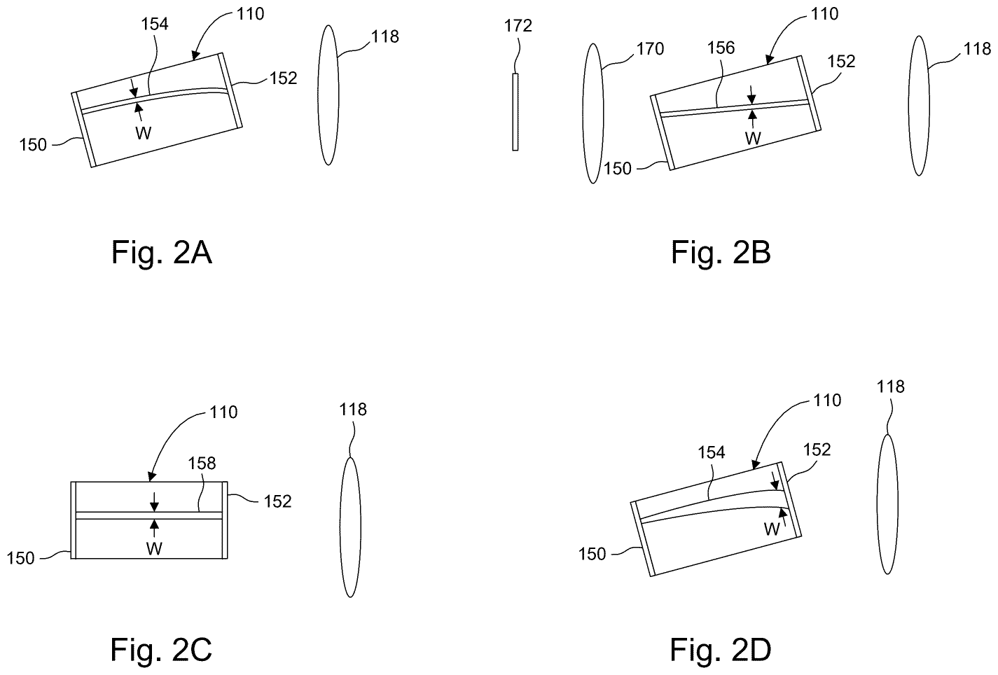

FIGS. 2A, 2B, 2C, and 2D illustrate several different chip architectures that are appropriate for the tunable laser 100 of FIG. 1. In some instances, additional components are required beyond those shown in FIG. 1 to create the laser operation, define the end of the laser cavity.

FIG. 2A shows a preferred gain chip architecture. This chip 110 is termed a single angled facet (SAF) edge-emitting chip. As such, it has a high reflectivity (HR) coated rear facet 150. It has an antireflective (AR) coated front facet 152. In addition, for improved performance, it has a curved ridge waveguide 154 that is perpendicular to the rear facet 150 but is angled at the interface with the front facet 152. This angling at the front facet along with the AR coating reduces reflections at the front facet reflectivity by up to 40 dB and significantly improves laser performance by reducing parasitic reflections that can otherwise lead to non-smooth tuning and mode-hopping.

FIG. 2B shows another potential edge-emitting gain chip configuration. The basic configuration is termed a semiconductor optical amplifier (SOA) gain chip. It has an AR coated rear facet 150 and an AR coated front facet 152. Its straight but angled ridge waveguide 156 intersects with the facets at an angle to minimize reflections back into the chip. In one example, its back facet light is coupled to a lens or pair of lenses 170 and a mirror 172 which reflects light to return through the lens and to the chip 110. The mirror could be made partially reflecting to additionally enable output from the back facet 150.

FIG. 2C shows another potential gain chip configuration. The basic configuration is termed a Fabry-Perot gain chip. As such, it preferably has an HR coated rear facet 150 and an AR coated front facet 152. The straight ridge waveguide 158 intersects with the front facet 152 at a perpendicular angle and thus does create some internal reflections that can affect performance.

FIG. 2D shows another gain chip architecture. This chip 110 is termed a flared or tapered single angled facet (SAF) edge-emitting chip. It has a high reflectivity (HR) coated rear facet 150. It has an antireflective (AR) coated front facet 152. In addition, the ridge is flared, widening in the direction of the front facet 152. When flared ridge widths are used, then the width W is the width as measured at the widest portion of the ridge.

According to the invention, widths of the ridges 154, 156, 158 of the chips shown in FIGS. 2A, 2B, 2C, and 2D are designed to support several spatial modes including lateral modes and/or transverse modes in their emissions.

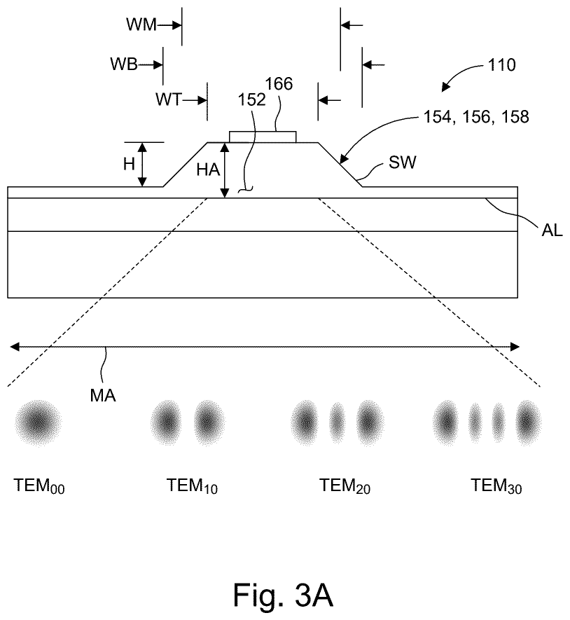

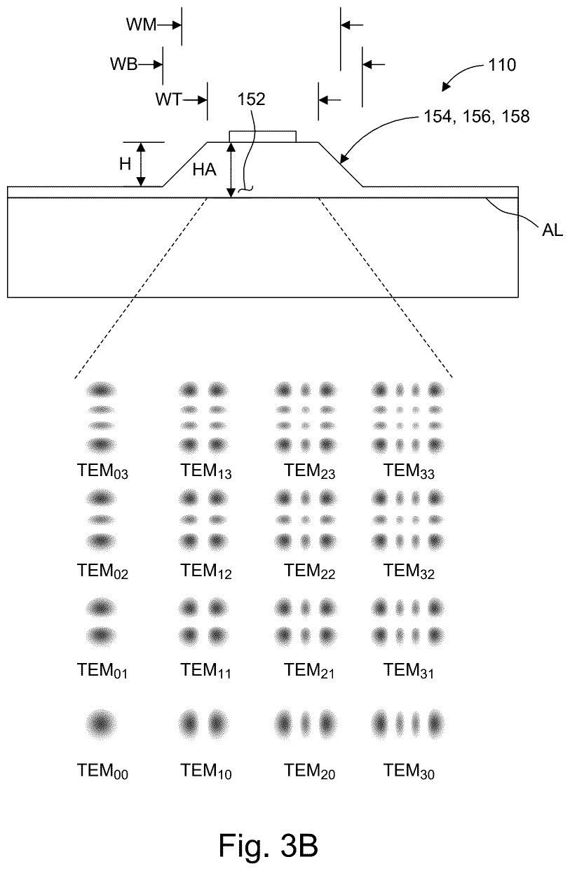

FIG. 3A shows the ridge profile at the chip's front facet 152 to support multiple lateral modes.

The ridge width W of the chip 110 is a crucial parameter that influences the optical confinement and principally the spatial mode structure. The ridge width is typically specified during the manufacturing process through photolithography, which in part defines the profile of the ridge waveguide transverse to the direction of the laser's waveguide.

On the other hand, the ridge height H and HA of the chip 110 is another important parameter that also influences the optical confinement and principally the transverse mode structure. In the figure, it is measured both as the distance HA between the active layer AL and the top of the ridge and the height H of the etched portion. The ridge heights H and HA are typically specified during the manufacturing process through the design of the epitaxial layers including the location of the active layer and the ridge height.

The ridge 154, 156, 158 of the chip 110 is formed using photolithographic techniques. A mask is used to define the area of semiconductor material that will be left standing in relief as the ridge. The width of the ridge is directly specified by the pattern on the mask. After photolithography, the wafer undergoes an etching process, such as reactive ion etching (RIE) or wet chemical etching, which removes the unprotected areas and leaves the ridge structure. The length of the etching process has a large impact on the ridge height. After etching, a conductive layer, usually a metal, is deposited on the top of the ridge to conduct the ridge injection current.

Preferably, the ridge 154, 156, 158 of the chip 110 is specified to support spatial lateral modes that are usually centered vertically around the chip's active layer AL.

Generally, the mode confinement and the effective index of the waveguide are determined by the waveguide's dimensions and the refractive index contrast between the ridge 154, 156, 158 and the surrounding material. The width that supports only a single lateral mode typically ranges from about 2 to 4 micrometers (μm) for GaAlAs lasers operating around 840 nm. Generally, for a GaAlAs laser emitting at 840 nm, a ridge width of approximately 3 μm is commonly used to ensure single-mode operation.

Therefore, in the preferred embodiment, the ridge width measured at the top of the ridge WT or at the base of the ridge WB or at a mid point WM along the side wall SW of the ridge is greater than 3 μm, such as WT and/or WM and/or WB is greater than 4 μm and preferably greater than 5 μm, and its greater than 8 μm in some examples.

At the wider ridge widths W, higher order lateral modes will simultaneously lase in addition to the TEM00 mode, such as the TEM10, TEM20, TEM30 modes. These modes will overlap each other, each being centered vertically on the active layer AL and centered laterally under the center of the ridge 154, 156, 158.

FIG. 3B shows the ridge profile at the chip's front facet 152 to support multiple transverse modes along with multiple lateral modes. This design is especially appropriate for full-field embodiments.

The ridge height H and HA, which impact the vertical confinement of the modes, usually range from 1 to 2 μm for a single spatial mode device. The exact heights depends on the layer structure of the GaAlAs laser diode and the specific goals for mode confinement and threshold current.

The ridge height H and HA of the chip 110 is a crucial parameter that influences the optical confinement and principally the transverse mode structure. The ridge height is typically specified during the manufacturing process through the etching process. If a wet etch is used, the etch time is the principal parameter.

In some embodiments, the ridge 154, 156, 158 of the chip 110 is specified to support multiple spatial transverse modes. Specifically, the ridge height H and the active layer ridge height HA, measured between the top of the ridge and the active layer AL, are greater than 2 μm, such as greater than 3 μm and possibly even greater than 4 or 5 μm.

With a wider and higher ridge, higher order lateral and transverse modes will simultaneously lase beyond the TEM00, such as the TEM_{NM}, wherein N, M=0, 1, 2, 3. These modes will overlap each other centered vertically on the active layer AL and centered laterally under the center of the ridge.

FIG. 4 shows the details of the interferometer 205 and its interfacing with the tunable laser 100 and line scan camera 228.

The free space beam 102 from the laser 100 is diverging. It is received by a mirror 310 mounted on a kinematic mount to a bench 308. The kinematic mount 310K minimally provides for adjusting the direction of the light in the x-y plane. In some examples, the kinematic mount 310K provides for adjusting the direction of the light in all three directions. This enables alignment of the beam for subsequent optics.

A series of components function as line-forming optics. They convert the light from the laser 100 into a line or more specifically a rectangular profile with an aspect ratio of at least 10 to 1 and typically greater than 100:1, and often 400:1, or more, measured at FWHM. That is, when looking along its optical axis, the light from the line-forming optics has a line or more specifically a high aspect ratio rectangular two-dimensional profile that is at least 10 times longer in along the z-axis than along y-axis, for example, measured at the FWHM.

A collimating lens 312 of the line-forming optics collimates the beam from mirror 310. Preferably collimating lens 312 is an achromat. This achromatic lens is designed to minimize the effects of chromatic aberration across the scan band of the laser 100. Chromatic aberration is a problem that occurs when different wavelengths of light are focused at different points, resulting in a blurry image. Currently, achromatic collimating lenses 312 uses two lenses made of different materials to correct for chromatic aberration over the scan band.

A neutral density filter 314 is provided to lower the power of the beam such as by lowering the power by 50% or more.

Next a cylindrical achromat lens 316 is provided to form the beam into a line. In the illustrated example, lens focuses the light in the x-y plane so that the line extends in the direction of the z-axis.

A cube beam splitter 318 next divides the laser light between a reference arm 320 and a sample arm 322.

In the reference arm 320, a reference arm mirror 324 is mounted to the bench 308 via a kinematic mount 324K. The reference arm mirror 324 is provided to fold the beam path and also allow for alignment. Next a cylindrical achromatic lens 326 collimates the beam. A reference arm neutral density filter 328 adjusts the power of the reference arm light and a reference arm mirror 330 is mounted on a linear motion rail 331 which in turn is mounted to the bench 308. The reference arm mirror 330 is moved on the linear motion rail 331 and moved by a linear motion actuator 331A to define and control the end of the reference arm and thus control of the delay to path match to the sample 202. Preferably the reference arm mirror 330 is mounted to the linear motion rail 331 via a kinematic mount 330K.

In the sample arm 322, a sample arm dichroic mirror 340 is held on a kinematic mount 340K, which is mounted on the bench 308. It folds the beam path and also allows for alignment by adjustment of its kinematic mount 340K. Light from a fixation target display 350 and to alignment camera 354 are transmitted through the dichroic mirror 340.

A telescope lens group 352 locates the fixation target 350 at infinity from the perspective of the patient's eye and its focus. The dichroic mirror 340 allows the green fixation target light to be transmitted to the patient and visible light from the patient to be transmitted to the alignment camera 354. Its advantage versus a long-pass dichroic is that the OCT beam is reflected instead of transmitted, which should avoid self-coherence and/or multireflection into the system. A 50/50 beamsplitter 356 couples light to the camera while transmitting light from the target to the patient.

In the sample arm, an achromat ocular lens 342 conditions the light so that the line is in focus on the retina, counteracting the eye's lens. The achromat ocular lens 342 is installed on a linear motion rail 341 and moved by a linear motion actuator 341A to adjust the lens position based on the patient's refractive error.

The light from the reference arm 320 and the sample arm 322 is combined in beamsplitter 318 and directed to the line scan or line field sensor 228. The linear array of the sensor 228 extends in the y-axis direction. A relay lens 360 is currently a triplet. This triplet is a Steinheil Triplet specifically, because it, with a single lens, provides a finite conjugate with relatively good aberration performance. A camera mirror 361 is mounted on a kinematic mount 361K to enable alignment of the interference beam to the line-scan camera 228.

For full-field operation, the cylindrical lens 316 used to form a line is replaced by spherical or afocal beam-expanding optics (e.g., Galilean or Keplerian expanders) and, optionally, a homogenizer to produce an areal, near flat-top illumination at the sample plane. The beam splitter 318 continues to divide light between the reference arm 320 and the sample arm 322 and recombines them onto the 2-D camera.

The reference arm 320 incorporates optics to match the numerical aperture and field of the sample arm, e.g., a beam expander and relay that image the reference pupil to the camera pupil. The sample arm 322 may retain the ocular lens 342 on rail 341 for ophthalmic correction, but configured so that a telecentric or Köhler-like illumination is achieved over the areal field.

A relay lens 360 (e.g., triplet or doublet pair) images the sample field onto the 2-D camera at the desired magnification while maintaining the field uniformity provided by the multimode source. The camera mirror 361 and mount 361K provide alignment of the areal interference pattern to the camera active area, analogous to the line-field alignment in FIG. 4.

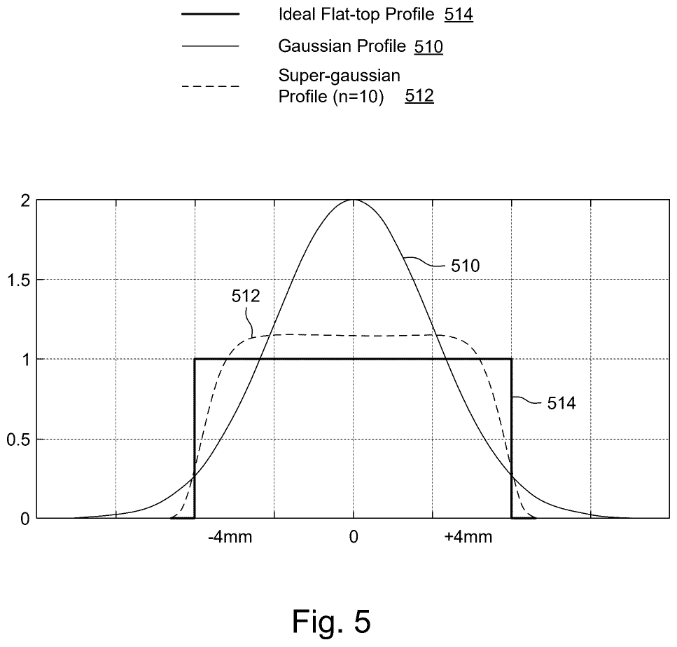

FIG. 5 is a plot of laser power along the major axis of the line as rendered on the retina 204 of the patient's eye 202. In a preferred embodiment, this line is at least 5 millimeters (mm) long, but is preferably longer since as 6 mm or more or 8 millimeters as shown. Still longer lines are possible such as greater than 10 mm or 12 mm or longer. For full-field embodiments, the plot applies to both axes of the light rendered on the eye 202.

In the case of the single longitudinal mode beam, the line would exhibit a Gaussian profile. This is suboptimal since at the center of the line there would be concern that optical power would exceed safety limits while insufficient power is provided near the edges at −/+4 mm for adequate signal to noise in the images.

In contrast, the present system supports multiple spatial modes that are distributed along the extent or major axis of the line projected onto the patient's retina. Therefore, the power distribution has a super Gaussian profile 512 that better approximates the ideal flat-top profile 514.

Preferably the line extent of the line 512 is parallel to the axis MA of the modes shown in FIG. 3A.

In addition, the existence of the multiple spatial modes reduces spatial coherence over the extent of the line, which reduces pixel cross talk.

In full-field embodiments, the areal irradiance on the sample is super-Gaussian in both lateral axes and approximates a 2-D flat-top. This reduces peak irradiance “hot spots” relative to a single-mode Gaussian beam and improves signal-to-noise uniformity across the field of view.

Representative metrics include, for example, ≤±20% intensity variation across the central 80% of the areal field and speckle contrast ≤0.5 on the 2-D camera owing to reduced lateral spatial coherence. (Values are exemplary and may be tuned by ridge geometry, fiber length, and aperture stops as discussed above.)

The orientation of the principal lateral mode axis MA shown in FIGS. 3A-3B is arbitrary with respect to the 2-D field and need not be aligned to camera axes; overlapping higher-order TEM_{N,M} modes from the chip collapse into the desired areal profile after field optics and optional multimode fiber propagation.

It should further be noted that while the description is specific to cat's eye laser architecture, the invention is relevant to other architectures such as other interference tuned lasers, tunable filter lasers, MEMS tunable lasers, and grating tuned lasers.

While this invention has been particularly shown and described with references to preferred embodiments thereof, it will be understood by those skilled in the art that various changes in form and details may be made therein without departing from the scope of the invention encompassed by the appended claims.

What is claimed is:

1. An optical coherence tomography (OCT) system comprising:
a swept optical source including a semiconductor gain chip configured during operation to lase in a plurality of spatial modes comprising at least one higher-order spatial mode beyond a fundamental mode;
coupling optics disposed between the swept optical source and an interferometer and configured to deliver output from the swept optical source to the interferometer while preserving the plurality of spatial modes; and
a free-space interferometer having free-space reference and sample arms, the interferometer being arranged to illuminate a sample and to combine light from the reference and sample arms for detection to generate interferometric signals from which depth-resolved information is reconstructed.

2. The system of claim 1, wherein the coupling optics comprise a free-space optical path from the swept optical source to the interferometer that maintains the plurality of spatial modes.

3. The system of claim 1, wherein the coupling optics comprise a multimode optical fiber between the swept optical source and the interferometer that maintains the plurality of spatial modes.

4. The system of claim 3, wherein the multimode optical fiber has a length of at least 1 m, optionally at least 10 m, and in some examples 40 m or more.

5. The system of claim 1, wherein the semiconductor gain chip is dimensioned to lase in multiple lateral modes, multiple transverse modes, or both.

6. The system of claim 5, wherein the gain chip comprises a ridge waveguide having a ridge width W greater than 3 μm, optionally greater than 5 μm, to support multiple lateral modes.

7. The system of claim 5, wherein the gain chip comprises a ridge waveguide having an active-layer ridge height H greater than 2 μm, optionally greater than 3 μm, to support multiple transverse modes.

8. The system of claim 1, further comprising a detector configured to receive interferometric light from the interferometer, the detector comprising at least one of: (i) a linear pixel array and (ii) a two-dimensional pixel array.

9. The system of claim 1, further comprising a controller configured to synchronize data acquisition to the optical sweep and to reconstruct OCT depth profiles from the detected interferometric signals.

10. The system of claim 1, wherein the swept optical source is configured to emit in a band centered about 840 nm with a tuning range of 30-100 nm.

11. The system of claim 1, wherein preserving the plurality of spatial modes produces a less-peaked illumination profile across an illuminated field relative to single-mode illumination and reduces lateral spatial coherence across the field to mitigate pixel cross-talk.

12. The system of claim 1, wherein the interferometer comprises a cube beam splitter that divides light between the free-space reference arm and sample arm and recombines light for detection.

13. The system of claim 1, wherein the detector and optics are arranged to form a line-field parallel OCT configuration in which a high-aspect-ratio line is projected onto the sample and sensed by a linear pixel array.

14. The system of claim 1, wherein the detector and optics are arranged to form a full-field parallel OCT configuration in which an areal field is projected onto the sample and sensed by a two-dimensional pixel array.

15. The system of claim 13, wherein the line has a length of at least 5 mm, optionally ≥6 mm, ≥8 mm, or ≥10 mm, and the intensity along the line is super-Gaussian relative to a Gaussian profile.

16. The system of claim 1, wherein the swept optical source further comprises an optical tuning element whose passband is varied during the sweep and a servo with an encoder configured to track a tuning curve for the optical sweep.

17. The system of claim 16, wherein the optical tuning element comprises a thin-film interference bandpass filter positioned in a collimated section of the cavity and tilt-tuned by the servo, the filter having a full-width at half-maximum bandwidth of 0.2-0.5 nm.

18. The system of claim 16, wherein the system further implements a synchronized power-versus-tuning-angle drive to flatten source output power across the sweep.

19. A method of optical coherence tomography comprising:
generating, with a swept optical source including a semiconductor gain chip configured to lase in a plurality of spatial modes comprising at least one higher-order spatial mode, a swept optical beam;
delivering the swept beam to a free-space interferometer via coupling optics that preserve the plurality of spatial modes;
dividing the beam into free-space sample and reference arms, combining light from the arms at a detector to produce interferometric signals; and
reconstructing depth-resolved information from the interferometric signals.

20. The method of claim 19, wherein the coupling optics comprise a multimode fiber of at least 10 m in length between the swept optical source and the free-space interferometer.

21. A swept optical source comprising:
a semiconductor gain chip configured during operation to lase in a plurality of spatial modes comprising at least one higher-order mode;
an optical tuning element configured to vary output optical frequency over a sweep; and
an output coupling arranged to deliver the output outside the laser cavity while preserving the plurality of spatial modes for free-space propagation or for injection into a multimode optical fiber;
or
An optical coupling apparatus configured to be arranged between a swept optical source and a free-space interferometer of an OCT system, the apparatus comprising:
at least one of (i) a free-space relay with numerical-aperture stops selected to pass higher-order spatial content from the source and (ii) a multimode optical fiber having a length of at least 1 m, and in some examples at least 10 m, arranged so that the plurality of spatial modes from the source are preserved to the interferometer while lateral spatial coherence is reduced; or
A full-field swept-source OCT system comprising: a free-space interferometer configured to illuminate an areal field on a sample and to combine sample-arm and reference-arm light on a two-dimensional camera; and a reference arm arranged to introduce at least one of (i) an off-axis tilt between reference and sample beams at the camera and (ii) phase-stepping via a driven reference mirror, whereby interferometric cross-terms are separable prior to k-space processing; or
An OCT system comprising:
a swept source; a free-space interferometer; and
a homogenizer disposed in a collimated section between the source and the interferometer, the homogenizer having a small diffusion angle selected to spatially mix the multimode field and reduce lateral coherence while maintaining the spectral linewidth required for axial OCT ranging.
